(19) United States
(12) Patent Application Publication
(10) Pub. No.: US 2019/0255210 A1
(43) Pub. Date: Aug. 22, 2019

(54) ANTIMICROBIAL COATING APPLIED TO RIGID STABILIZING COMPONENTS OF ORTHOPEDIC EQUIPMENT

(71) Applicant: Parasol Medical, LLC, Buffalo Grove, IL (US)

(72) Inventor: Patrick E. Eddy, Allendale, MI (US)

(73) Assignee: Parasol Medical, LLC, Buffalo Grove, IL (US)

(21) Appl. No.: 16/269,947

(22) Filed: Feb. 7, 2019

Related U.S. Application Data
(60) Provisional application No. 62/631,728, filed on Feb. 17, 2018.

(51) Int. Cl. A61L 15/14; A61F 5/01; A61F 5/02; A61L 15/12

(52) U.S. Cl. CPC A61L 15/14; A61F 5/0106; A61F 5/0111; A61L 2420/06; A61L 15/125; A61L 2300/404; A61F 5/02

(57) ABSTRACT

A knee ligament support device comprising a lower portion, an upper portion, a rigid stabilizer member coupled to the lower portion and the upper portion, and an antimicrobial coating. The lower portion is configured to surround a portion of a leg below a knee. The upper portion is configured to surround a portion of the leg above the knee. The rigid stabilizer member is configured to stabilize movement of the knee. The rigid stabilizer member has an exterior surface. The antimicrobial coating is applied to the exterior surface of rigid stabilizer member. The antimicrobial coating includes a silane quaternary ammonium ion or salt thereof. The antimicrobial coating is applied to the exterior surface of the rigid stabilizer member as a solution including the silane quaternary ammonium ion or salt thereof and a solvent. The solvent can be isopropyl alcohol.

### CROSS-REFERENCE TO RELATED APPLICATION

This patent application claims, pursuant to 35 U.S.C. § 119(e), priority to and the benefit of U.S. Provisional Patent Application No. 62/631,728, entitled “ANTIMICROBIAL COATING APPLIED TO RIGID STABILIZING COMPONENTS OF ORTHOPEDIC EQUIPMENT,” filed on Feb. 17, 2018, the entire disclosure of which is incorporated herein by reference.

### BACKGROUND OF THE INVENTION

An athlete sometimes wears orthopedic equipment on various parts of the athlete's body. The orthopedic equipment provides stability to the body part to which it is attached, which may prevent injury. The orthopedic equipment can have soft, fabric-constructed, members, which are wrapped around the body part to attach the orthopedic equipment to the body part. In addition, the orthopedic equipment sometimes includes a rigid member that adds stability to the body part, to help prevent the body part, for example, from rotating, pivoting, or moving too far in a certain direction that could cause injury. However, orthopedic equipment, including the rigid members thereof, can harbor harmful microbes, bacteria, and viruses that could subsequently become transferred to another person or the athlete wearing the orthopedic equipment and thus cause illness.

### BRIEF SUMMARY OF THE INVENTION

According to a first aspect of the present disclosure, a knee ligament support device comprises: a lower portion configured to surround a portion of a leg below a knee; an upper portion configured to surround a portion of the leg above the knee; a rigid stabilizer member coupled to the lower portion and the upper portion, and configured to stabilize lateral movement of the knee; the rigid stabilizer member comprising an exterior surface; and an antimicrobial coating applied to the exterior surface of rigid stabilizer member; wherein, the antimicrobial coating includes a silane quaternary ammonium ion or salt thereof.

Embodiments of the first aspect of the invention can include any one or a combination of the following features:

the silane quaternary ammonium ion or salt thereof is one or more of:
3-(trimethoxysilyl)propyldimethyloctadecyl ammonium ion,
3-(trimethoxysilyl)propyldimethyloctadecyl ammonium chloride,
3-(trihydroxysilyl)propyldimethyloctadecyl ammonium ion, or
3-(trihydroxysilyl)propyldimethyloctadecyl ammonium chloride;
   the antimicrobial coating is applied to the exterior surface of the rigid stabilizer as a solution including the silane quaternary ammonium ion or salt thereof and a solvent;
   the solvent is isopropyl alcohol;
   the isopropyl alcohol is between 30 percent to 90 percent by weight of the solution;
   the silane quaternary ammonium ion or salt thereof is between 0.1 percent and 10 percent by weight of the solution;
   the silane quaternary ammonium ion or salt thereof is between 1.9 percent and 2.1 percent by weight of the solution; and
   the isopropyl alcohol is between 55 percent and 65 percent by weight of the solution.

According to a second aspect of the present disclosure, an ankle support device comprises: a rigid stabilizer portion configured to stabilize lateral movement of an ankle of a leg, the rigid stabilizer portion comprising an exterior surface; padding attached to the rigid stabilizer portion configured to be disposed between the rigid stabilizer portion and the leg; one or more straps or banding configured to secure the rigid stabilizer portion and padding to the leg; and an antimicrobial coating applied to the exterior surface of rigid stabilizer portion; wherein, the antimicrobial coating includes a silane quaternary ammonium ion or salt thereof.

Embodiments of the second aspect of the invention can include any one or a combination of the following features:

the silane quaternary ammonium ion or salt thereof is one or more of:
3-(trimethoxysilyl)propyldimethyloctadecyl ammonium ion,
3-(trimethoxysilyl)propyldimethyloctadecyl ammonium chloride,
3-(trihydroxysilyl)propyldimethyloctadecyl ammonium ion, or
3-(trihydroxysilyl)propyldimethyloctadecyl ammonium chloride;
   the antimicrobial coating is applied to the exterior surface of the rigid stabilizer portion as a solution including the silane quaternary ammonium ion or salt thereof and a solvent;
   the solvent is isopropyl alcohol;
   the isopropyl alcohol is between 30 percent to 90 percent by weight of the solution;
   the silane quaternary ammonium ion or salt thereof is between 0.1 percent and 10 percent by weight of the solution;
   the silane quaternary ammonium ion or salt thereof is between 1.9 percent and 2.1 percent by weight of the solution; and
   the isopropyl alcohol is between 55 percent and 65 percent by weight of the solution.

According to a third aspect of the present disclosure, a back support device comprises: a wrap portion configured to surround a torso including at least part of a back; a vertical rigid stabilizer portion coupled to the wrap portion, and configured to stabilize movement of the back; the vertical rigid stabilizer portion comprising an exterior surface; and an antimicrobial coating applied to the exterior surface of the vertical rigid stabilizer portion; wherein, the antimicrobial coating includes a silane quaternary ammonium ion or salt thereof.

Embodiments of the third aspect of the invention can include any one or a combination of the following features:

the silane quaternary ammonium ion or salt thereof is one or more of:
3-(trimethoxysilyl)propyldimethyloctadecyl ammonium ion,
3-(trimethoxysilyl)propyldimethyloctadecyl ammonium chloride,
3-(trihydroxysilyl)propyldimethyloctadecyl ammonium ion, or
3-(trihydroxysilyl)propyldimethyloctadecyl ammonium chloride;
   the antimicrobial coating is applied to the exterior surface of the vertical rigid stabilizer portion as a solution including the silane quaternary ammonium ion or salt thereof and a solvent;
   the solvent is isopropyl alcohol;
   the isopropyl alcohol is between 30 percent to 90 percent by weight of the solution;
   the silane quaternary ammonium ion or salt thereof is between 1.9 percent and 2.1 percent by weight of the solution; and
   the isopropyl alcohol is between 55 percent and 65 percent by weight of the solution.

These and other features, advantages, and objects of the present invention will be further understood and appreciated by those skilled in the art by reference to the following specification, claims, and appended drawings.

### BRIEF DESCRIPTION OF THE DRAWINGS

FIG. 1 is a perspective view of a knee ligament support device, with a knee shown in phantom, illustrating a rigid stabilizer member including an exterior surface;

FIG. 2 is a magnified view of area II of FIG. 1, illustrating an antimicrobial coating applied over the exterior surface of the rigid stabilizer member of FIG. 1;

FIGS. 3A and 3B are perspective views of an ankle support device, with the ankle and leg shown in phantom, illustrating a rigid stabilizer portion including an exterior surface;

FIG. 4 is a magnified view of area IV of FIG. 3A, illustrating the antimicrobial coating applied over the exterior surface of the rigid stabilizer portion of FIG. 3A;

FIG. 5 is a perspective view of a back support device, with a torso including a lower back shown in phantom, illustrating a vertical rigid stabilizer portion including an exterior surface; and

FIG. 6 is a magnified view of area VI of FIG. 5, illustrating the antimicrobial coating applied over the exterior surface of the vertical rigid stabilizer portion of FIG. 5.

### DETAILED DESCRIPTION

For purposes of description herein, it is to be understood that the specific devices and processes illustrated in the attached drawings, and described in the following specification are simply exemplary embodiments of the inventive concepts defined in the appended claims. Hence, physical characteristics relating to the embodiments disclosed herein are not to be considered as limiting, unless the claims expressly state otherwise.

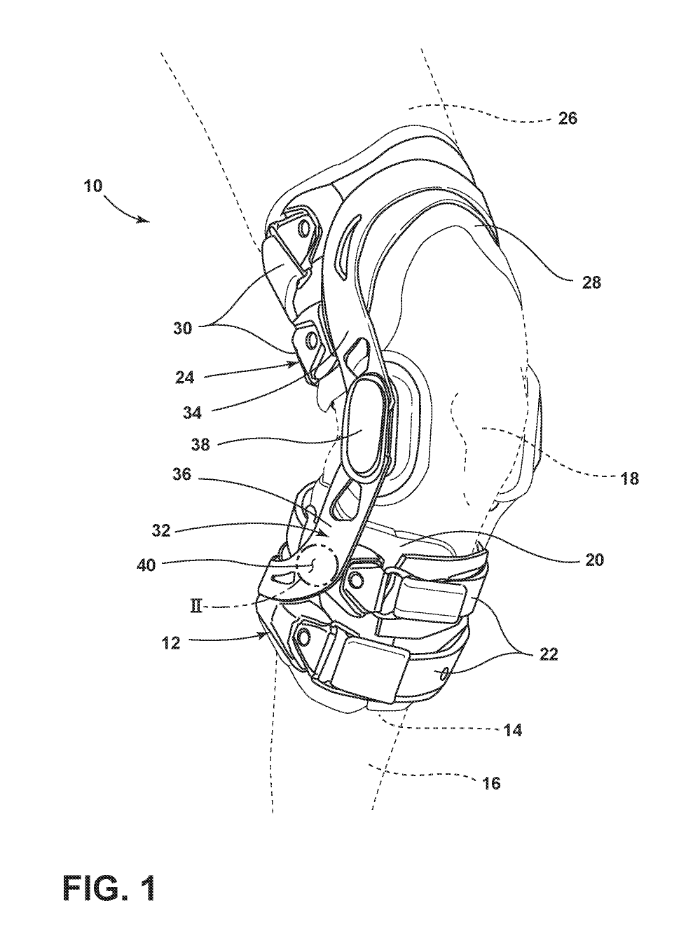

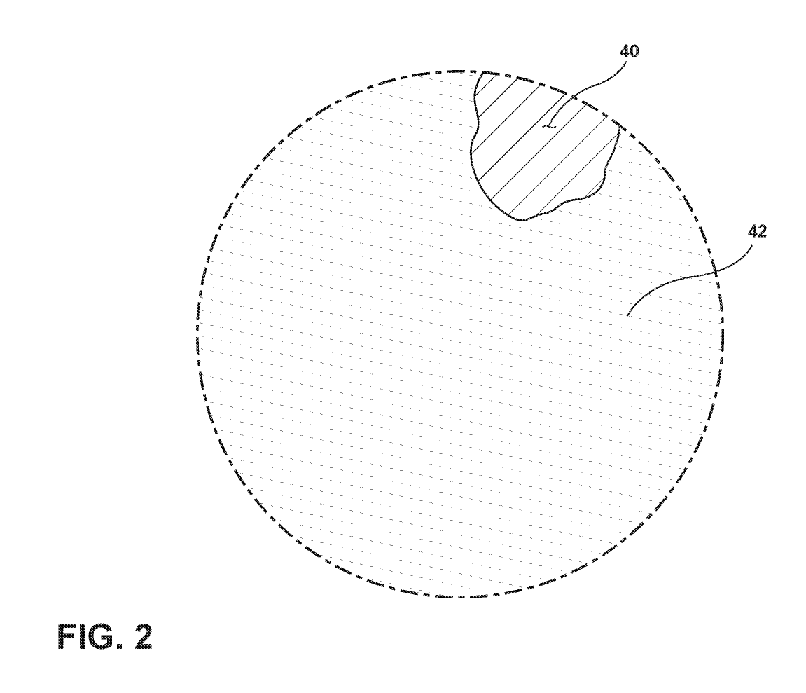

Referring to FIGS. 1-2, a knee ligament support device 10 includes a lower portion 12. The lower portion 12 is configured to surround a portion 14 of a leg 16 below a knee 18. For example, the lower portion 12 can include a fabric leg-contact portion 20 and one or more straps 22 attached to the fabric leg-contact portion 20 to attach the lower portion 12 to the portion 14 of the leg 16 below the knee 18. The knee ligament support device 10 further includes an upper portion 24 above the lower portion 12. The upper portion 24 is configured to surround a portion 26 of the leg 16 above the knee 18. For example, the upper portion 24 can likewise include a fabric leg-contact portion 28 and one or more straps 30 attached to the fabric leg-contact portion 28 to attach the upper portion 24 to the portion 26 of the leg 16 above the knee 18. The knee ligament support device 10 further includes a rigid stabilizer member 32. The rigid stabilizer member 32 can be made at least in part from metal and/or a rigid plastic, such as an upper metal component 34 and a lower metal component 36, coupled together by a metal or plastic coupler 38. The rigid stabilizer member 32, depending on the positioning relative to the knee 18, can reduce or prevent lateral or medial movement of knee 18 or hyperextension thereof, and thus is configured to stabilize movement of the knee 18. The rigid stabilizer member 32 is coupled to the lower portion 12 and the upper portion 24. The rigid stabilizer member 32 has an exterior surface 40, which is a rigid, non-fibrous surface, such as that provided by metal or plastic.

The knee ligament support device 10 further includes an antimicrobial coating 42 applied to at least a portion of the exterior surface 40 of the rigid stabilizer member 32, including the upper metal component 34, the lower metal component 36, and the coupler 38. FIG. 2 specifically illustrates the antimicrobial coating 42 applied over the exterior surface 40 of the lower metal component 36, but it should be understood that the antimicrobial coating 42 can be applied over any exterior surface 40 of the rigid stabilizer member 32, including the exterior surfaces 40 of the upper metal component 34 and coupler 38.

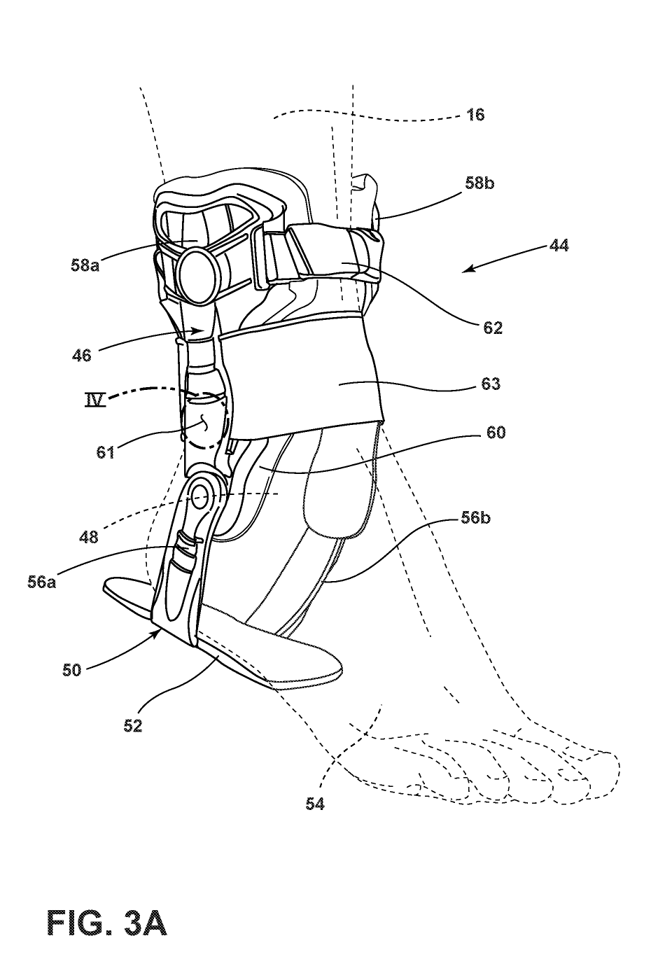

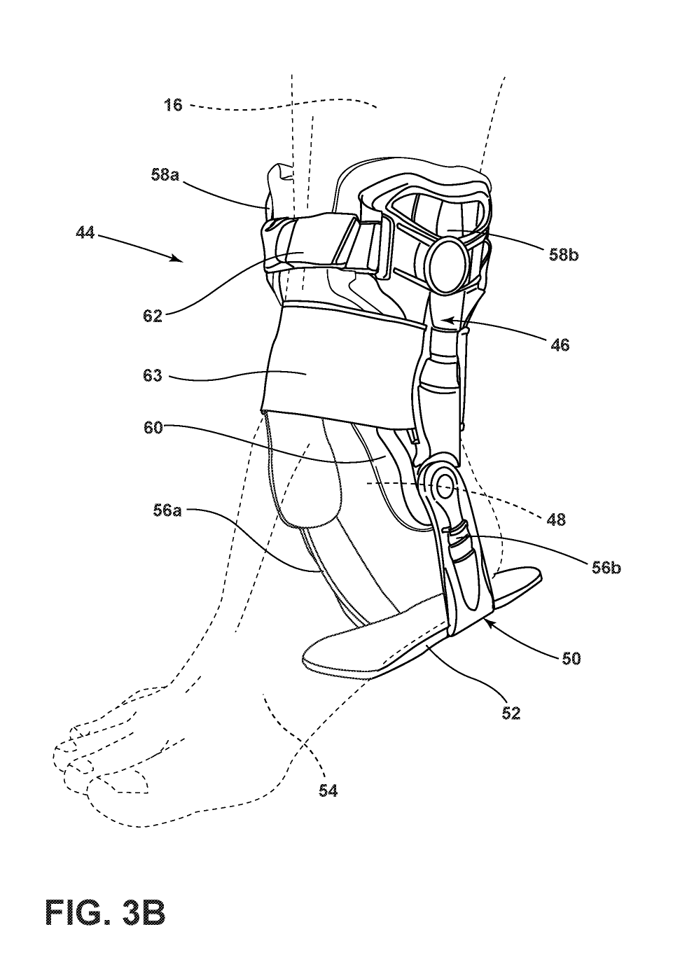

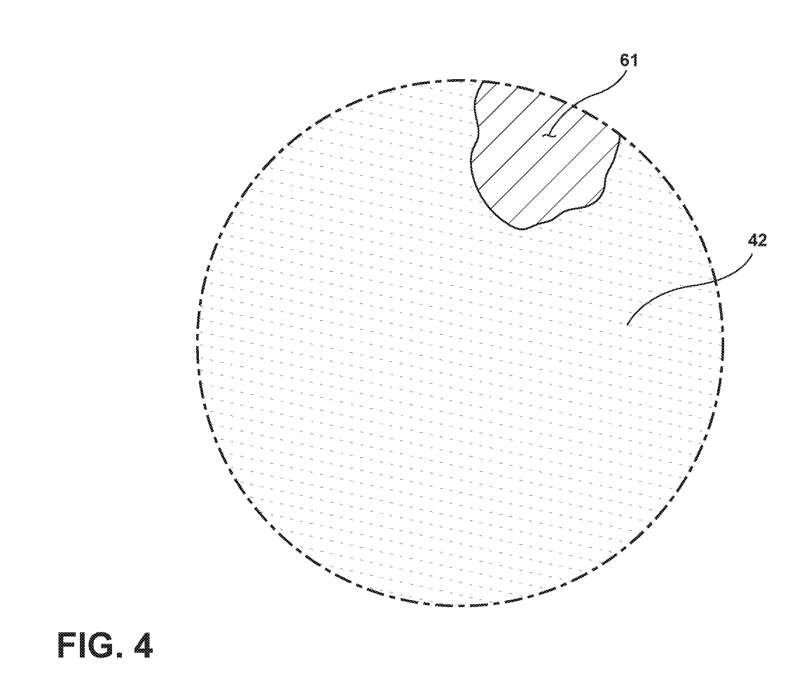

Referring now to FIGS. 3A, 3B, and 4, an ankle support device 44 includes a rigid stabilizer portion 46. The rigid stabilizer portion 46 is configured to stabilize lateral movement of an ankle 48 of the leg 16. In this embodiment, the rigid stabilizer portion 46 has a lower member 50 with a foot section 52 configured to be disposed under a foot 54 connected to the leg 16 and two opposing upwardly extending members 56a, 56b extending from the sides of the foot section 52. Each of the upwardly extending members 56a, 56b are pivotally coupled to an upper side member 58a, 58b, respectively, which are generally disposed above the ankle 48 when worn. The upwardly extending members 56a, 56b and the upper side members 58a, 58b, because they are rigid, prevent the ankle 48 from bending/pivoting sideways.

The ankle support device 44 further includes padding 60. The padding 60 is attached to the rigid stabilizer portion 46 and is disposed between the rigid stabilizer portion 46 and the leg 16, when the ankle support device 44 is worn. The padding 60 can be covered by a fabric. The ankle support device 44 further includes a strap 62 and banding 63. The strap 62 and banding 63 can be attached to the padding 60 or the rigid stabilizer portion 46, and secure the rigid stabilizer portion 46 and padding 60 to the leg 16. The rigid stabilizer portion 46 has an exterior surface 61, which is a rigid, non-fibrous surface, such as that provided by metal or plastic.

The ankle support device 44 further includes the antimicrobial coating 42 applied to the exterior surface 61 of the rigid stabilizer portion 46, including the upwardly extending members 56a, 56b and upper side members 58a, 58b. FIG. 4 specifically illustrates the antimicrobial coating 42 applied over the exterior surface 61 of the upwardly extending member 56a, but it should be understood that the antimicrobial coating 42 can be applied over any exterior surface of the rigid stabilizer portion 46, including the exterior surfaces 61 of the upper side members 58a, 58b and the upwardly extending member 56b.

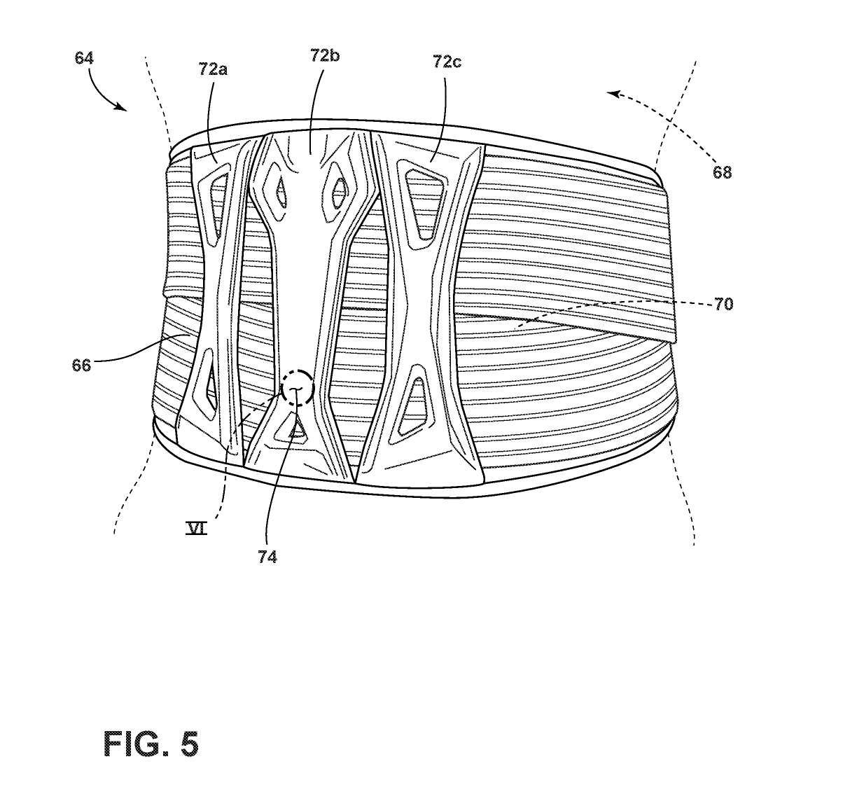

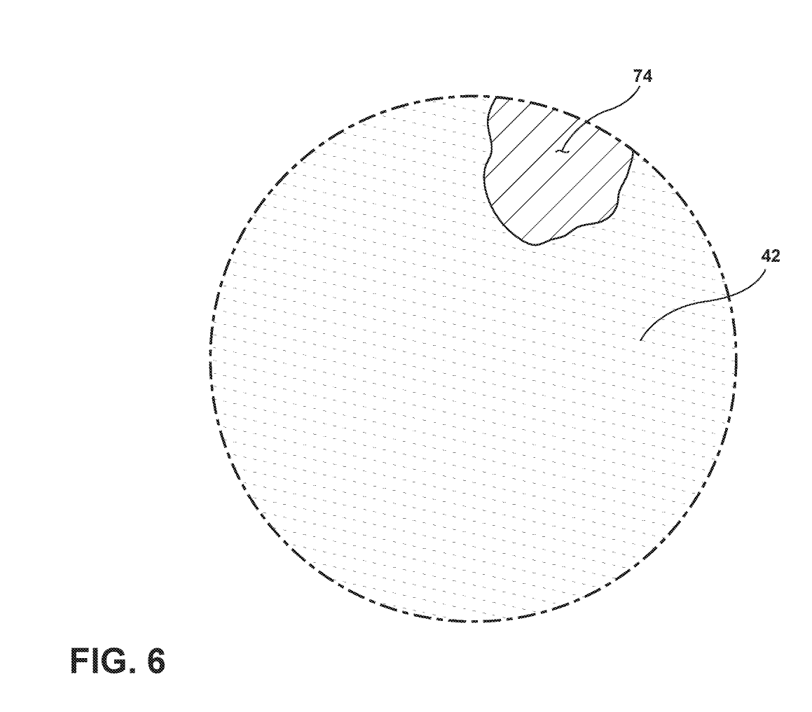

Referring now to FIGS. 5 and 6, a back support device 64 includes a wrap portion 66. The wrap portion 66 is configured to surround a torso 68 including at least part of a lower back 70. The wrap portion 66 could be elastic fabric. The back support device 64 further includes several vertical rigid stabilizer portions 72a-72c. The vertical rigid stabilizer portions 72a-72c are coupled to the wrap portion 66. The vertical rigid stabilizer portions 72a-72c are configured to stabilize movement of the lower back 70, such as to lessen the ability of the lower back 70 to bend backward, in order to prevent lower back 70 strain or other injury. The vertical rigid stabilizer portion 72b includes an exterior surface 74, which is a rigid, non-fibrous surface, such as that provided by metal or plastic.

The back support device 64 further includes the antimicrobial coating 42 applied to the exterior surface 74 of the vertical rigid stabilizer portions 72a-72c, including vertical rigid stabilizer portion 72b. FIG. 6 specifically illustrates the antimicrobial coating 42 applied over the exterior surface 74 of the vertical rigid stabilizer portion 72b, but it should be understood that the antimicrobial coating 42 can be applied over any exterior surface 74 of the vertical rigid stabilizer portions 72a-72c, including the exterior surfaces 74 of the vertical rigid stabilizer portions 72a and 72c.

The antimicrobial coating 42 includes a silane quaternary ammonium ion or salt thereof. Among the preferable silane quaternary ammonium ions or salts thereof include 3-(trimethoxysilyl)propyldimethyloctadecyl ammonium ion, 3-(trimethoxysilyl)propyldimethyloctadecyl ammonium chloride, 3-(trihydroxysilyl)propyldimethyloctadecyl ammonium ion, and 3-(trihydroxysilyl)propyldimethyloctadecyl ammonium chloride, and the silane quaternary ammonium ion or salt thereof can be one or more of those listed above.

The antimicrobial coating 42 can be applied to the exterior surface 40 of the rigid stabilizer member 32 of the knee ligament support device 10, the exterior surface 61 of the rigid stabilizer portion 46 of the ankle support device 44, and the exterior surface 74 of the vertical rigid stabilizer portion 72b of the back support device 64 as a solution that include the silane quaternary ammonium ion or salt thereof and a solvent. The solvent evaporates after the solution is applied to the exterior surfaces 40, 61, 74, leaving the silane quaternary ammonium ion or salt thereof on the exterior surfaces 40, 61, 74. A preferred solvent is isopropyl alcohol.

The silane quaternary ammonium ion or salt thereof can comprise between 0.1 percent and 10 percent by weight of the solution. More preferably, the silane quaternary ammonium ion or salt thereof can comprise between 0.75 percent and 5 percent by weight of the solution. Even more preferably, the silane quaternary ammonium ion or salt thereof can comprise between 1.9 percent and 2.1 percent by weight of the solution.

As for the isopropyl alcohol, the isopropyl alcohol can comprise between 30 percent to 90 percent by weight of the solution. More preferably, the isopropyl alcohol can comprise between 55 percent and 65 percent by weight of the solution. An example preferable solution comprises (by weight) 60.0 percent isopropyl alcohol, 2.02 percent 3-(trimethoxysilyl)propyldimethyloctadecyl ammonium chloride, and 34.19 percent deionized water. Applying the solution forms the antimicrobial coating over the exterior surfaces 40, 61, 74 and inhibits the growth of microbes, viruses, bacteria, and the like on those exterior surfaces 40, 61, 74.

The solution can be applied via spraying the solution containing the silane quaternary ammonium ion or salt thereof and the solvent with an electrostatic sprayer. Alternatively, the solution can be applied with a wipe soaked with the solvent, a spray bottle containing the solvent, and other means. The solvent of the solution generally evaporates, leaving the antimicrobial coating 42 with the silane quaternary ion or salt thereon, which imparts the antimicrobial properties to the exterior surfaces 40, 61, 74 to which the antimicrobial coating 42 is applied.

It is to be understood that variations and modifications can be made on the afore-mentioned structure without departing from the concepts of the present invention, and further it is to be understood that such concepts are intended to be covered by the following claims unless these claims by their language expressly state otherwise.

The invention claimed is:

1. A knee ligament support device comprising:
a lower portion configured to surround a portion of a leg below a knee;
an upper portion configured to surround a portion of the leg above the knee;
a rigid stabilizer member coupled to the lower portion and the upper portion, and configured to stabilize movement of the knee;
the rigid stabilizer member comprising an exterior surface; and
an antimicrobial coating applied to the exterior surface of rigid stabilizer member;
wherein, the antimicrobial coating includes a silane quaternary ammonium ion or salt thereof.

2. The knee ligament support device of claim 1,
wherein, the silane quaternary ammonium ion or salt thereof is one or more of:
3-(trimethoxysilyl)propyldimethyloctadecyl ammonium ion,
3-(trimethoxysilyl)propyldimethyloctadecyl ammonium chloride,
3-(trihydroxysilyl)propyldimethyloctadecyl ammonium ion, or
3-(trihydroxysilyl)propyldimethyloctadecyl ammonium chloride.

3. The knee ligament support device of claim 2,
wherein, the antimicrobial coating is applied to the exterior surface of the rigid stabilizer member as a solution including the silane quaternary ammonium ion or salt thereof and a solvent.

4. The knee ligament support device of claim 3,
wherein, the solvent is isopropyl alcohol.

5. The knee ligament support device of claim 4,
wherein, the isopropyl alcohol is between 30 percent to 90 percent by weight of the solution.

6. The knee ligament support device of claim 5,
wherein, the silane quaternary ammonium ion or salt thereof is between 0.1 percent and 10 percent by weight of the solution.

7. The knee ligament support device of claim 6,
wherein, the silane quaternary ammonium ion or salt thereof is between 1.9 percent and 2.1 percent by weight of the solution; and
the isopropyl alcohol is between 55 percent and 65 percent by weight of the solution.

8. An ankle support device comprising:
a rigid stabilizer portion configured to stabilize lateral movement of an ankle of a leg, the rigid stabilizer portion comprising an exterior surface;
padding attached to the rigid stabilizer portion configured to be disposed between the rigid stabilizer portion and the leg;
one or more straps or banding configured to secure the rigid stabilizer portion and padding to the leg; and
an antimicrobial coating applied to the exterior surface of rigid stabilizer portion;
wherein, the antimicrobial coating includes a silane quaternary ammonium ion or salt thereof.

9. The ankle support device of claim 8,
wherein, the silane quaternary ammonium ion or salt thereof is one or more of:
3-(trimethoxysilyl)propyldimethyloctadecyl ammonium ion,
3-(trimethoxysilyl)propyldimethyloctadecyl ammonium chloride,
3-(trihydroxysilyl)propyldimethyloctadecyl ammonium ion, or
3-(trihydroxysilyl)propyldimethyloctadecyl ammonium chloride.

10. The ankle support device of claim 9,
wherein, the antimicrobial coating is applied to the exterior surface of the rigid stabilizer portion as a solution including the silane quaternary ammonium ion or salt thereof and a solvent.

11. The ankle support device of claim 10,
wherein, the solvent is isopropyl alcohol.

12. The ankle support device of claim 11,
wherein, the isopropyl alcohol is between 30 percent to 90 percent by weight of the solution.

13. The ankle support device of claim 12,
wherein, the silane quaternary ammonium ion or salt thereof is between 0.1 percent and 10 percent by weight of the solution.

14. The ankle support device of claim 13,
wherein, the silane quaternary ammonium ion or salt thereof is between 1.9 percent and 2.1 percent by weight of the solution; and
the isopropyl alcohol is between 55 percent and 65 percent by weight of the solution.

15. A back support device comprising:
a wrap portion configured to surround a torso including at least part of a lower back;
a vertical rigid stabilizer portion coupled to the wrap portion, and configured to stabilize movement of the back;
the vertical rigid stabilizer portion comprising an exterior surface; and
an antimicrobial coating applied to the exterior surface of vertical rigid stabilizer portion;
wherein, the antimicrobial coating includes a silane quaternary ammonium ion or salt thereof.

16. The back support device of claim 15,
wherein, the silane quaternary ammonium ion or salt thereof is one or more of:
3-(trimethoxysilyl)propyldimethyloctadecyl ammonium ion,
3-(trimethoxysilyl)propyldimethyloctadecyl ammonium chloride,
3-(trihydroxysilyl)propyldimethyloctadecyl ammonium ion, or
3-(trihydroxysilyl)propyldimethyloctadecyl ammonium chloride.

17. The back support device of claim 16,
wherein, the antimicrobial coating is applied to the exterior surface of the vertical rigid stabilizer portion as a solution including the silane quaternary ammonium ion or salt thereof and a solvent.

18. The back support device of claim 17,
wherein, the solvent is isopropyl alcohol.

19. The back support device of claim 18,
wherein, the isopropyl alcohol is between 30 percent to 90 percent by weight of the solution.

20. The back support device of claim 19,
wherein, the silane quaternary ammonium ion or salt thereof is between 1.9 percent and 2.1 percent by weight of the solution; and
the isopropyl alcohol is between 55 percent and 65 percent by weight of the solution.
